(19) United States
(12) Patent Application Publication
(10) Pub. No.: US 2022/0328146 A1
(43) Pub. Date: Oct. 13, 2022

(54) COMPUTERIZED SCREENING TOOL FOR BEHAVIORAL HEALTH

(71) Applicant: THE RESEARCH INSTITUTE AT NATIONWIDE CHILDREN'S HOSPITAL, Columbus, OH (US)

(72) Inventor: Rajeev KRISHNA, Columbus, OH (US)

(21) Appl. No.: 17/639,847

(22) PCT Filed: Sep. 4, 2020
(86) PCT No.: PCT/US2020/049390

§ 371 Date: Mar. 2, 2022

Related U.S. Application Data
(60) Provisional application No. 62/895,667, filed on Sep. 4, 2019.

(51) Int. Cl. G16H 10/60; G16H 50/20

(52) U.S. Cl. CPC G16H 10/60; G16H 50/20

(57) ABSTRACT

A system including a database, a healthcare provider interface and a respondent interface for assisting in the selection, administration and scoring of measurement tools to be provided to respondents. The database includes a plurality of diagnostic domains, respondent infomation for respondents, and measurement tool, data objects. The measurement tool data objects identify measurement tools and include conditional operators to select a subset of the measurement tools. The health care provider interface is configured to accept a selection of a diagnostic domain. The respondent interface includes measurement tool selection logic and scoring logic. The database is configured to provide a measurement tool data object based on the selected domain and a respondent type. The system evaluates the measurement tool data object using additional respondent information to select at least one measurement tool. The respondent interface administers the selected measurement tools to respondents and to scores completed measurement tools.

### RELATED APPLICATIONS

This application claims the benefit of U.S. Application 62/895,667, filed Sep. 4, 2019, and which is incorporated by reference.

### BACKGROUND

Standard measurement tools (questionnaires) exist for assessing a patient's mental health status. These tools can supplement the assessment by a mental health provider, and offer consistent, validated results across time points. Examples of such measurement tools include: Level 1 DSM5 (cross-cutting symptom screen) for parents and children; PHQ9P (depression screen) for parents and child; Vanderbilt (ADHD screen) for parents; Child Mania Rating Scale (for mania, an indication of bipolar disorder) for parents; and SCARED (anxiety screen) for parent and child, among others.

These measurement tools may have different questionnaires for adult and child patients, parents, teachers, and others involved in the life of child patients. There may also be several measurement tools for any given behavioral health issue. For example, measurement tools to assess impairment from mental health issues include but are not limited to. Difficulties in Emotion Regulation (DERS) (impairment due to emotions questionnaire) for parent and child; OHIO Scales (impairment screening) for parent and child; and Columbia Impairment Scales (impairment screening) for parent.

Some of these measurement tools may be public domain and some may be proprietary. The measurement tools may, vary in scope and detail.

Traditionally, a health care professional would select a measurement tool and provide a paper copy to a patient or parent, then score the completed measurement tool. Lately, there has been a push for improvement and automation of the process of administration, collection and scoring of patient-completed measurement tools, as well as interest in tracking patient outcomes to improve delivery of care.

Known attempts to automate patient completed measurement tools include administering measurement tools by computer and scoring the measurement tools automatically, with the goal of producing a complete report of results before the patient sees the doctor or healthcare professional. One common model involves a fully automated system which conducts a high-level screening and determines which measurement tools are needed based on a computer algorithm (e.g., to assess anxiety or depression). Another common model involves making the full set of tools available to the provider, and allowing manual selection of the appropriate tools for any given respondent. A combination of these approaches is also frequently used, with an initial set of assessments selected by algorithm, with subsequent manual custom refinement. There are notable disadvantages to both approaches. The fully automated approach excludes the skill, knowledge and experience of the health care provider. The manual selection approach puts too much burden on a given health care provider, who may have limited time, and be tasked with making decisions outside his or her field of expertise. For example, a mental health specialist (e.g.: a psychiatrist) may diagnose an area of concern and select appropriate measurement tools, but a primary care provider may not know which measurement tool to use, or the psychometric properties and meaning of different tools within that area of concern. This may result in the selection of a measurement tool of too little or too much detail, or inappropriate interpretation of the tools selected. Similar patients may thus receive different care for the same condition from different providers, undermining any potential benefits of standardization across the care continuum. The manual selection and assignment process, particularly when multiple respondents are involved (e.g.: patient, parent, and teacher) can also be time consuming to a very busy provider.

What is needed is a system that automatically selects and administers appropriate context-specific health measurement tools, but which is guided by the assessment skills of the healthcare provider, and knowledge of appropriate tool usage derived from domain experts.

### SUMMARY

A system for assisting in the selection, administration and scoring of measurement tools to be provided to respondents is provided. The system includes a database, a healthcare provider interface and a respondent interface. The database is configured with a plurality of diagnostic domains, respondent information for a plurality of respondents, and a plurality of measurement tool data objects, the measurement tool data objects each identifying a plurality of measurement tools and including conditional operators to select a subset of the measurement tools. The health care provider interface has access to the database and is configured to accept a selection of a diagnostic domain by a health care provider. The respondent interface has access to the database and is configured to receive respondent information from the one or more respondents. The respondent interface also includes measurement tool selection logic and scoring logic.

The database is configured to provide a measurement tool data object based on the selected domain and a respondent type. The system is further configured to evaluate the measurement tool data object using additional respondent information to select at least one measurement tool, In some embodiments, the measurement tool selection logic in the respondent interface evaluates the measurement tool data object with respect to the respondent information. The respondent interface is further configured to administer the selected at least one measurement tool to the respondent and to score completed measurement tools with the scoring logic. The measurement tool data object may be stored in the database as a CLOB.

The domain may be selected from a list of mental health categories. In another embodiment, the domain may be selected from physical health domains. In other embodiments, the domain may be selected from any potential categorization scheme that results in a specific set of patient/respondent self-reported outcome measures, including mental health, physical health, psychosocial assessments, high risk behaviors, etc. The respondent type may be selected from a list comprising patient, parent, guardian, and educator. The additional respondent information may comprise patient status. The patient status may comprise a selection between new or returning patient status.

The database may comprise a plurality of tables, including table including the plurality of diagnostic domains, a table including the respondent information, and a table including the plurality of measurement tool data objects. The database may be further configured to provide a measurement tool data object based on a healthcare department or a location in which a respondent is being evaluated. The system may be configured to provide a new measurement tool data object automatically upon a change in patient location.

The system may be configured to provide scored measurement tool results to a health care provider via the health care provider interface.

The database may be further configured with packages of department-specified measurement tools which override the selection logic.

### BRIEF DESCRIPTION OF THE DRAWINGS

FIG. 1 is a block diagram of a system according to one aspect of the present invention.

FIG. 2 is an example of a Users database table according to another aspect of the present invention.

FIG. 3 is an example of a Diagnosis Group database table according to another aspect of the present invention.

FIG. 4 is an example of a Respondent Type database table according to another aspect of the present invention,

FIG. 5 is an example of a Respondent Map database table according to another aspect of the present invention.

FIG. 6 is an example of a Department database table according to another aspect of the present invention.

FIG. 7 is an example of a Department Class database table according to another aspect of the present invention.

FIG. 8 is an example of a Diagnosis Metric Map database table according to another aspect of the present invention.

FIG. 9 is a flow chart according to another aspect of the present invention.

### DETAILED DESCRIPTION

A system is provided which guides a health care provider though the process of selecting measurement tools to be applied to respondents based on a domain as identified by a health care provider or caregiver, a type of respondent who is responding to the measurement tool, and a context of where in the health care process the measurement tool is being administered. For example, depression, anxiety, and mania may be considered different domains. A respondent may be any individual answering assessments, and may include the patient, the patient's parents or other primary caregivers, and other related persons, such as school teachers. The context may include where in the health care process the patient is being seen, such as a physician's office, an emergency room, a general clinic, or a specialty clinic, and what stage of diagnosis or treatment, such as initial diagnosis, more in-depth diagnosis, or outcome tracking.

Once a domain of concern is identified by a health care provider, a computer system determines what measurement tools are to be applied within that provider-selected domain. For example, a health care provider could instruct the system to screen a patient for social anxiety, and the system would determine what measurement tool or tools to apply to screen for or measure a level of anxiety. The domains are not limited to the examples herein, such as mental health diagnosis and tracking, and may extend to other behavioral health domains and physical health domains. The selection of appropriate tools for a given domain and stage of care may be determined administratively, allowing experts in both the domain and in the measurement tools themselves to drive the process. Given a domain of concern, the system can automatically determine appropriate tools for different respondent types, dramatically lowering the workload on the individual healthcare provider.

The selection of one or more measurement tools is based on the domain and context. The selection process may be implemented in a database decision matrix. As set forth above, the domain is provider selected. The decision matrix receives as inputs the patient, additional respondents answering assessments, such the patient's caregivers, educators, etc. (if applicable), where the patient is being seen and what stage of diagnosis or treatment. For example, a public domain measurement tool may be indicated as an initial screening in a general clinic, but a more detailed measurement tool may be indicated if the patient has already been diagnosed and referred to a specialty clinic.

In some embodiments, the measurement tools are administered by and automatically scored by computer. Results may be automatically entered into an electronic healthcare record system.

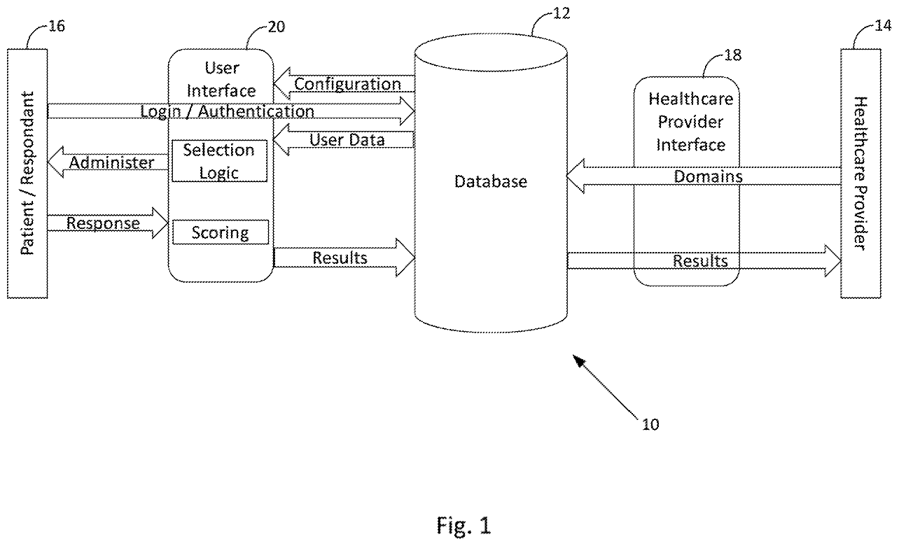

In one example illustrated in FIG. 1, the system 10 is implemented in part in a relational database 12. A plurality of healthcare provider departments 14 and, a plurality of respondents 16 may access the database 12 through appropriate interfaces, For example, in some embodiments, healthcare providers 14 may designate domains and receive results via a healthcare provider user interface 18. Patients and respondents 16 may receive measurement tools and provide responses via a respondent user interface 20. The respondent user interface 20 receives respondent data and configuration information from, the relational database 12. The respondent user interface includes measurement tool selection logic 22 and measurement tool scoring logic 24, and provides measurement tool scoring results to the database 12.

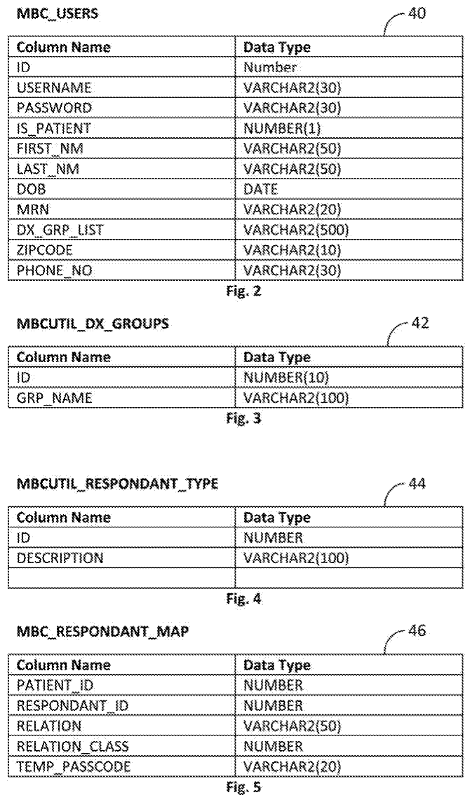

A USERS table 40 of the database (FIG. 2) may include fields for basic information concerning respondents using the system, such as name, date of birth, and contact information. The USERS table also includes a field for designating whether the respondent is a patient. For respondents designated as patients, a Diagnosis Group List field is populated with one or more Diagnosis Domain IDs associated with a particular patient. The USERS table also includes a unique User ID is assigned to each respondent. These fields are populated when a respondent is initially added to the system, and may be available at all subsequent contexts of treatment.

The Diagnosis Domain IDs may be associated with a DIAGNOSIS GROUP table 42 (FIG. 3). Each record in the DIAGNOSIS GROUP table may include a field for a Diagnosis ID number and a Diagnosis Group Name. The Diagnosis Group Name field is populated with the diagnosis domains for assessment or measurement. In the case of mental health, examples of Diagnosis Group Name domains may include Depression, Mania, ADHD, ADHD-Hyperactive, ADHD-Inattentive, Anxiety, Anxiety-Generalized, Anxiety-School Avoidance, Anxiety-Social, etc. In other fields of healthcare, other diagnosis domains may be used. In some embodiments, a health care provider selects a domain for a user from a list of domains in the Diagnosis Group Name fields. Optionally, a default domain may also be provided to accommodate patients for which no prior diagnosis (if any) is known or made by a health care professional.

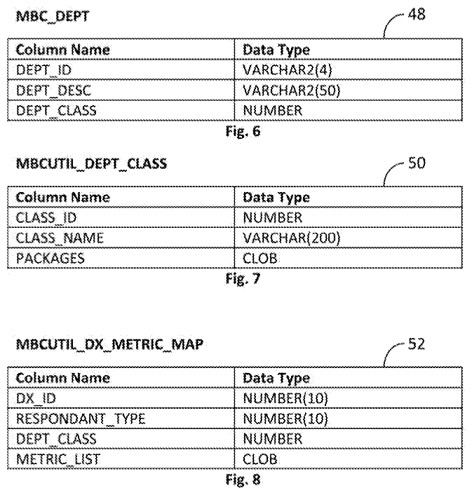

A RESPONDENT MAP table 46 (FIG. 5) may be provided to associate respondents in the system. The RESPONDENT MAP table may include a Patient 10 field and a Despondent ID field. A record exists for each user in which the Respondent ID field is assigned the corresponding ID number from the USERS table. For a given patient, the Patient ID field and a Respondent ID would be the same (the patient is also the respondent). More than one respondent may be associated with each patient, and in this case, the Patient ID field would be assigned the patient's ID number from the USERS table. A Relation field for a relationship, between a patient and a respondent may be included, providing information such as patient, mother, etc. A Relation Class field for the type of relationship may also be provided. For example, the relations of mother, father, and legal guardian to a patient may all be assigned to the same Relation Class, primary caregiver. The Relation Class field may be linked to a RESPONDENT TYPE table 44 (FIG. 4). The RESPONDENT TYPE table includes an ID number for the relation's class and description of possible respondent types, including patient, primary caregiver, school teacher, etc.

By maintaining data in this relationship, the system can use knowledge about a given respondent's association with the patient, and the diagnostic domain groups assigned to that patient, to assist in determining which assessments to administer to a particular respondent. This structure is also what allows new respondent who have never been seen in the system before to be assigned the correct assessments. If a new caregiver comes in with the patient for a particular visit, the act of establishing them in the system and setting them up as a caregiver for that patient create all of the necessary relationships to allow the system to identify which assessments are appropriate for them.

The type of respondent is necessary in order to delineate between measurement tools for different populations. For example, a particular measurement tool may have a different format for the patient versus for a parent versus for a school teacher. Similarly, the “department class” identifying the location of the respondent allows the system to respond to differences in location details. This allows, for example, for a specialty trauma clinic to have a patient complete a much more detailed trauma assessment then they would receive for the exact same diagnostic domain (“trauma”) in a general therapy clinic.

A DEPARTMENT table 48 (FIG. 6) may be provided with fields for a unique Department ID, Department Description, and Department Class. Department Description fields are populated with a name and/or physical location of a department. The Department Class field may be populated with a Department Class ID. The Department Class ID field may be linked to a DEPARTMENT CLASS table 50 (FIG. 7), which includes records having fields for Department Class ID, Department Class Name, and Packages associated with a given Department Class (where appropriate). Department Classes may include, for example, Psychiatry-General, Inpatient-General, inpatient-Psychiatric, Therapy-General, etc. In the illustrated embodiment, the Packages are stored as Character Large Objects (CLOBs). Other database field types may also be used. The Package Objects may be used to describe a set of assessments that can be manually selected by staff at a given department, and overrides the automated selection logic of the system. For example, a package for a General Psychiatry department may include a chief complaint, review of systems, PHQ9, and SCARED measurement tools for new patients, while a package for a General Therapy department would include only a chief complaint, PHQ9, and SCARED measurement tools for new patients. This provides a convenient method of overriding the selection logic for more customized demands for special patient populations that may not yet fit into the capabilities of the selection logic itself.

A DIAGNOSIS METRIC MAP table 52 (FIG. 8) may link the Respondent Type, Diagnosis Group and Department Class to the actual Measurement Tools. This DIAGNOSIS METRIC MAP table has records with fields for Respondent Type, Diagnosis Group ID (i.e., domain), Department Class, and Metric List. Various combinations of values in these fields map to a specific list of measurement tools that are indicated to be administered. In the illustrated example, the list of measurement tools is embodied in a Metric List data object which is stored as a CLUB. The Metric List data objects allow for further conditionality. For example, for a given Diagnosis Group ID (0, default), Respondent Type (1, patient), and Department Class ID (0, default), the system would evaluate a Metric List data object with conditional operators which would select a different list of measurement tools depending on whether the visit involved a new patient (np), a new patient to this department type but known to the system (rv_new), a standardized return visit assessment if the provider has not selected relevant domains (rv_std), and a standard set of assessments that should be included regardless of other domains selected (rv_def). In another example, while one of the entries below for depression is “PHQ9”, the entry may be further formatted to include age-related conditionality such as “{tend: agemin:8; set: “PHQ9”}”. This is a string form of a JavaScript object (in JSON notation), which indicates that the measurement system should administer the PHQ9 to the parent, but only if the patient age is 8 or greater. Conditionality may also be based on time since the previous administration of the measurement tool. Additional conditionality logic may be added to the data objects as needed.

This arrangement allows for an enormous degree of flexibility in how measurement tools are selected For example, through a combination of different department classes, and different measurement tools with specific sub conditions, the system may be configured such that a patient arriving in an emergency department is given a set of measurement tools that are immediately useful to that department. If that patient is moved to a medical bed and admitted, an additional set of measurement tools can be automatically administered. This additional set of measurement tools excludes any measurement tools already administered in the emergency department, but otherwise includes them and adds further detail to the picture. If this patient is subsequently admitted to an inpatient psychiatric unit from the medical bed, a further set of measurement tools can be completed that would further supplement the assessment without overlapping with recently completed measurement tools.

If that patient then receives a set of diagnostic domains while on the inpatient unit and transitions to an outpatient office visit but is brought to that visit by a different parent than the one who was involved in the inpatient stay, an entirely different set of measurement tools that are appropriate to the outpatient environment, but also appropriate to the specific diagnostic concerns for that patient would be automatically enabled, including for the new respondent (the new parent) who the system had never seen before. All of this is managed by setting up the appropriate data structures in the) background, with essentially no added work beyond the basic clinical decision making on providers, and a highly customized and appropriate set of measurement tools for the patient and family.

At the same time, this flexibility and adaptability does not come at the cost of increased database complexity, For example, combining multiple different patient statuses with conditional logic in a single Metric List data object avoids having to create, link and maintain multiple different records in a table for each potential patient status. Also, new conditional operations may be added without having to modify the database schema.

The present system is not limited to selecting and/or administering questionnaires relevant to the mental health care field. Questionnaires may also be assigned and scored in other fields of health care, especially at the patient intake stage. The system may also be adapted to identify other diagnostic or measurement tools, such as self-reported medical diagnostic assessments.

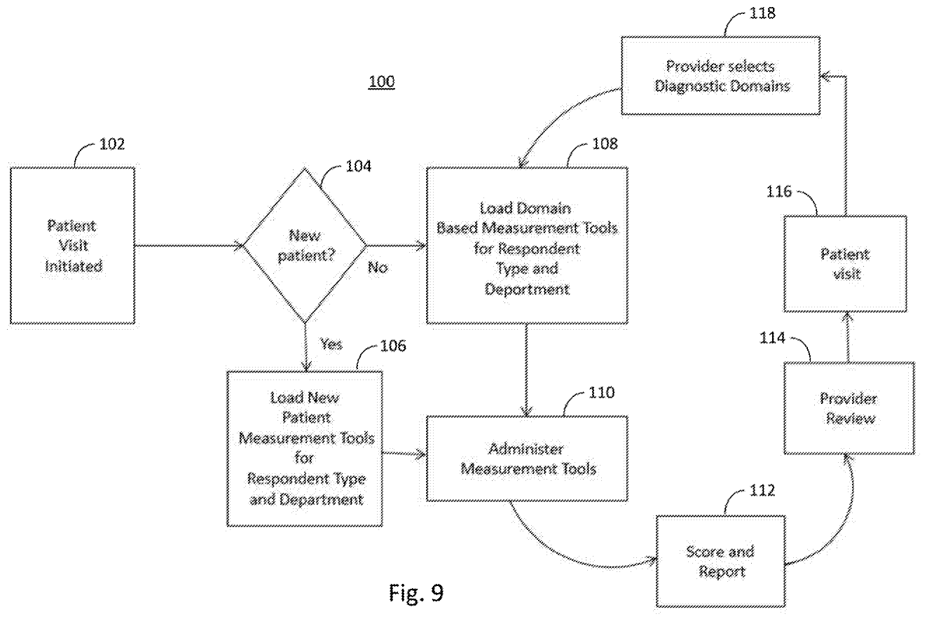

A flow chart of a method 100 for using the system is provided in FIG. 9. In the first step, a patient visit is initiated 102. The system checks whether the patient is a new patient in step 104. If the user is a new patient, new patient measurement tools for the appropriate respondent type and department are loaded in step 106. If the patient is a returning patient, domain-based measurement tools for the appropriate respondent type and department are loaded in step 108. The measurement tools are administered in step 110, and scored and reported in step 112. A healthcare provider reviews the scored report in step 114 and visits with the patient in step 116. Based on this, the healthcare provider selects or modifies the selected diagnostic domains of concern in step 118. This input, based on the clinical knowledge and skill, of the provider, is then used to drive the next stage of the cycle, ensuring continued consistent and reliable assessments with minimal time and efficiency burdens on all individuals involved.

Several advantages may be realized with the invention. The system takes into account the health care provider's insights into a given patient while reducing the amount of time and the level of skill required of the provider. Even if the provider is highly skilled and knowledgeable in the particular diagnostic domain, the system lessens the burden on the provider and reduces the chances for clerical errors. The measurement tools to be used fora given situation may be coordinated and consistently applied. This will improve consistency of diagnosis and outcome tracking.

In the foregoing specification, the invention has been described with reference to specific exemplary embodiments thereof, Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A system for assisting in the selection, administration and scoring of measurement tools to be provided to respondents, comprising:
a database configured with a plurality of diagnostic domains, respondent information for a plurality of respondents, and a plurality of measurement tool data objects, the measurement tool data objects each identifying a plurality of measurement tools and including conditional operators to select a subset of the measurement tools;
a health care provider interface having access to the database and configured to accept a selection of a diagnostic domain by a health care provider;
a respondent interface having access to the database, the respondent interface being configured to receive respondent information from the one or more respondents and including measurement tool selection logic and scoring logic;
the database being configured to provide a measurement tool data object based on the selected domain and a respondent type; and
the system being configured to evaluate the measurement tool data object using addition& respondent information to select at least one measurement tool;
wherein the respondent interface is further configured to administer the selected at least one measurement tool to the respondent and to score completed measurement tools with the scoring logic.

2. The system of claim 1, wherein the domain is selected from a list of mental health categories.

3. The system of claim 1, wherein the respondent type is selected from a list comprising patient, parent, guardian, and educator.

4. The system of claim 1, wherein the additional respondent information comprises patient status.

5. The system of claim 4, wherein the patient status comprises a selection of new or returning patient status.

6. The system of claim 1, wherein the database comprises a plurality of tables, including table including the plurality of diagnostic domains, a table including the respondent information, and a table including the plurality of measurement tool data objects.

7. The system of claim 1, wherein the measurement tool selection, logic in the respondent interface evaluates the measurement tool data object with respect to the respondent information.

8. The system of claim 1, further configured to provide scored measurement tool results to a health care, provider via the health care provider interface.

9. The system of claim 1, wherein the database is further configured to provide a measurement tool data object based on a healthcare department in which a respondent is being evaluated.

10. The system of claim 1, wherein the database is further configured to provide a measurement tool data object based on a location in which a respondent is being evaluated.

11. The system of claim 10, wherein the system is configured to provide a new measurement tool data object automatically upon a change in patient location.

12. The system of claim 1, wherein the database is further configured with packages of depart. specified measurement tools which override the selection

13. The system of claim 1, wherein the measurement tool data object is stored in the database as a

14. The system of claim 1, wherein the diagnostic domains include physical health domains.
